(19) United States
(12) Patent Application Publication
(10) Pub. No.: US 2019/0365730 A1
(43) Pub. Date: Dec. 5, 2019

(54) ANAESTHETIC COMPOSITION COMPRISING ROPIVACAINE, PRILOCAINE AND LIDOCAINE

(71) Applicant: Med Solutions Sweden Aktiebolag, Stockholm (SE)

(72) Inventor: Mohamed Abdel-Rehim, Stockholm (SE)

(21) Appl. No.: 16/485,099

(22) PCT Filed: Feb. 9, 2018
(86) PCT No.: PCT/SE2018/050117

§ 371 Date: Aug. 9, 2019

(30) Foreign Application Priority Data
Feb. 9, 2017 (SE) 1750121-4

(51) Int. Cl. A61K 31/445; A61K 31/573; A61K 9/00; A61K 31/167; A61P 23/02; A61K 31/245

(52) U.S. Cl. CPC A61K 31/445; A61K 31/573; A61K 9/0019; A61K 9/0014; A61P 23/02; A61K 31/245; A61K 31/167

(57) ABSTRACT

The present invention relates to pharmaceutical compositions for alleviation of pain and/or skin inflammation comprising (i) ropivacaine and (ii) two or more locoregional anesthetics of amide type and optionally hydrocortisone, to methods of preparing such compositions and uses thereof.

### FIELD OF THE INVENTION

The present invention relates to pharmaceutical compositions for alleviation of pain and/or skin inflammation comprising (i) ropivacaine and (ii) two or more locoregional, anesthetics of amide type, to methods of preparing such compositions and uses thereof.

### BACKGROUND

Anesthetics of the amide type are administered to a body for locoregional reversible inhibition of pain. Topical anesthetics of the amide type are locally applied to skin and commonly used for reversible inhibition of nociceptive pain.

Lidocaine or xylocaine belongs to this class of substances and is used to numb tissues in a specified area, to treat ventricular tachycardia, and also for use in nerve blocking.

Bupivacaine is another substance classified as topical anesthetics of an amide type and was discovered in 1957. After bupivacaine was found to be associated with cardiac arrest the topical anesthetics ropivacaine was developed.

Ropivacaine is an anesthetic of the amide type, having a long duration time, as well as anti-inflammatory and anti-bacterial effects compared to lidocaine. Ropivacaine has been found to have less cardiotoxicity than bupivacaine in animal models and ropivacaine hydrochloride is today marketed by Astrazeneca under the trade name Naropin.

Prilocaine is yet another local anesthetic of the amino amide type and in its injectable form, it is often used in dentistry under the trademark name Citanest.

Several mixtures of topical anesthetics are currently in use in clinical practice to achieve synergistic and additive therapeutic effects of the individual components. EMLA, lidocaine 2.5% and prilocaine 2.5% by weight (w/w, i.e 25 mg/g), is approved, indicated and recommended for pre-surgical anesthesia of normal intact skin. The current use of EMLA requires application of the medicated cream to normal intact skin under (i.e. plastic tape such as tegaderm) for 1-2 hours prior to surgery and a duration time of about one hour is usually observed.

Despite the widely use of mixtures of local anesthetics in clinical practice there is still a need for a topical anesthetic composition having improved efficacy. In particular, a topical composition with minimal side effects providing effective anesthesis is to be a preferred alternative treatment compared to strong medications having adverse systemic effects and adverse side effects or needle injections associated with anxiety, fear, discomfort, and pain.

Additionally, lidocaine, bupivacaine, ropivacaine and priolocain are used as active agents for epidural pain relief. Thus all of the substances mentioned above may be used for regional anesthesia, in addition to topical anesthesia. However, due to for instance the association of bupivacaine with cardiac arrest, bupivacaine may not be suitable for such pain relief.

Thus, there is furthermore a need for a more efficient composition to be used topically or for injection or infusion, in order to enable longer surgical intervention time or to achieve a better postoperative analgesia.

### SUMMARY

Today ropivacaine solution is commercialized by AstraZeneca under brand name “Naropin®” (see above). Additionally many researchers have investigated a combination of different local anaesthetics such as lidocaine with ropivacaine. In 2014, a research group from Romania investigated the use of lidocaine and ropivacaine solution and they found good results (the analgesic effect increased by 2 fold) when a combination of lidocaine and ropivacaine was used instead of ropivacaine alone (Lazăr A., Szederjesi J., Copotoiu R., Copotoiu S-M and Azamfirei L; Acta Medica Marisiensis (2014), 60 (2), 41-43). In another work which was published in 2003 by Volchkov et al. (Volchkov V A, Didur M D, Strashnov V I, Anesteziologiia i Reanimatologiia, 2003 (4):25-28) a combination of local anesthetics with promedol and corticosteroids was tested and they concluded that: “The most prolonged analgesic effect was registered in groups, whose patients received bupivacaine or ropivacaine, which prevented the onset of pain before the anti-inflammatory effect of corticosteroids started”.

It is well known that bupivacaine is more toxic than prilocaine, lidocaine or ropivacaine (Åkerman B., I.-B. Hellberg I. B., Trossvik C., Acta Anaesthesiologica Scandinavica, Vol 32, Issue 7, 1988, 571-578). Thus the use of lidocaine, prilocaine or ropivacaine instead of bupivacaine is safer and will result in long lasting anesthesia.

It is thus an object of the present application to provide compositions for longer lasting and safer anesthesia, both locally and regionally. In the present disclosure, “locoregional” is used to define the anesthesia to be provided. Locoregional means that the anesthesia is restricted to a localized region of the body, and thus only eliminates pain in for instance the region of surgery. Within the present disclosure, locoregional anesthesia also comprises local anesthesia.

Thus, there is provided a pharmaceutical composition for alleviation of pain and/or skin inflammation comprising:

(i) ropivacaine, and

(ii) two or more locoregional, anesthetics of amide type.

There is also provided a pharmaceutical composition as described herein for use as a medicament in therapy.

There is also provided a pharmaceutical composition as described herein for use in the treatment and/or prevention of at least one of pain, inflammation, bacterial infection, skin irritation.

There is also provided a pharmaceutical composition as described herein for the manufacture of a medicament for the treatment and/or prevention of at least one of pain, inflammation, bacterial infection, skin irritation.

There is also provided a method for treatment and/or prevention of at least one of pain, inflammation, bacterial infection, skin irritation comprising administering to a mammal, such as a human or an animal, in need thereof an effective amount of a pharmaceutical composition as described herein.

There is also provided pharmaceutical delivery system comprising the pharmaceutical composition as described herein, wherein said delivery system is selected from the group consisting of cream, lotion, emulsion preparation, topical liquid, aerosol solution, gel, transdermal delivery system in the form of a patch, jelly, ointment, spray, liposome delivery system and liposome delivery system. Said delivery system may also be selected from the group comprising infusion solutions, epidural solutions and injection solutions.

Embodiments are specified in the ensuing description and in the dependent claims.

### Abbreviations Used

ATC code—Anatomical Therapeutic Chemical Classification System.

ATC code NO1—subgroup of the classification system part of the anatomical group nervous system, N.

CAS number—a unique numerical identifier assigned by Chemical Abstracts Service to every substance described in the open scientific literature.

Hrs, hours

Bupivacaine—the substance 1-Butyl-N-(2,6-dimethylphenyl)-2-piperidinecarboxamide CAS Number 73360-54-0, ATC code NO1BB01

EMLA—a mixture of equal quantities by weight of lidocaine and prilocaine, a 5% emulsion preparation, containing 2.5% each of lidocaine and prilocaine

Hydrocortisone—the substance 11β, 17α,21-Trihydroxypregn-4-ene-3,20-dione, 17-Hydroxycorticosterone, cortisol, CAS Number 50-23-7, ATC code A01AC03

Lidocaine—the substance 2-Diethylamino-N-(2,6-dimethylphenyl)acetamide,

CAS Number 137-58-6, ATC code C01BB01

Mepivacaine—the substance 1-Methyl-2′,6′-pipecoloxylidine hydrochloride, N-(2,6-Dimethylphenyl)-1-methyl-2-piperidinecarboxamide hydrochloride

CAS Number 1722-62-9, ATC code N01BB03

Prilocaine—the substance N-(2-Methylphenyl)-2-(propylamino)propanamide hydrochloride

CAS Number 1786-81-8, ATC code N01BB04

Ropivacaine—the substance (S)-N-(2,6-dimethylphenyl)-1-propylpiperidine-2-carboxamide hydrochloride monohydrate

CAS Number 132112-35-7, ATC code N01BB09

Tetracaine—the substance 4-(Butylamino)benzoic acid 2-(dimethylamino)ethyl ester

CAS Number 94-24-6, ATC code C05AD02

Dibucaine (also known as cinchocaine)—the substance 2-butoxy-N-[2-(diethylamino)ethyl]quinoline-4-carboxamide

CAS Number 85-79-0, ATC code N01BB06/C05AD04

Articaine—the substance methyl 4-methyl-3[2-(propylamino)propanoylamino]thiophene-2-carboxylate, CAS Number 23964-58-1, ATC code N01BB08

Etidocaine—the substance N-(2,6-dimethylphenyl)-2-[ethyl(propyl)amino]butanamide

CAS number 36637-18-0, ATC code N01BB07

Levobupivacain—the substance (2S)-1-butyl-N-(2,6-dimethylphenyl)piperidine-2-carboxamide,

CAS number 27262-47-1, ATC code N01BB10

Trimecaine—the substance 2-(diethylamino)-N-(2,4,6-trimethylphenyl)acetamide CAS number 616-68-2,

In this document, it will be appreciated that lidocaine, mepivacaine, prilocaine, ropivacaine, tetracaine, dibucaine, articaine, etidocaine, levobupivacaine, and/or trimecaine as described herein may be present as a therapeutically acceptable salt and/or solvate. Thus, these compounds are not limited to the hydrochloride salts and solvents described herein.

### DETAILED DESCRIPTION

In accordance with the present invention there is provided a pharmaceutical composition comprising:

(i) ropivacaine, and

(ii) two or more locoregional anesthetics of amide type.

In this document, the term “two or more locoregional, anesthetics of amide type” is understood not to include ropivacaine.

The two or more local anesthetics of amide type may be selected from a group consisting of bupivacaine, lidocaine, mepivacaine, prilocaine, tetracaine, dibucaine, articaine, etidocaine, levobupivacaine, and trimecaine.

Additionally or alternatively, the pharmaceutical composition may further comprise hydrocortisone so that it comprises:

(i) ropivacaine, and

(ii) two or more locoregional, anesthetics of amide type and hydrocortisone.

The pharmaceutical composition described herein may comprise from about 1% by weight to about 5% by weight of ropivacaine and from about 1% by weight to about 5% by weight of the two or more locoregional, anesthetics of amide type.

In an example, the pharmaceutical composition described herein may comprise about 2.5% by weight of ropivacaine, about 2.5% by weight of prilocaine and about 2.5% by weight of lidocaine.

The pharmaceutical composition described herein may be a topical pharmaceutical composition. The pharmaceutical composition described herein may also be a solution for epidural injection of for infusion.

There is also provided a pharmaceutical composition as described herein for use as a medicament in therapy.

There is also provided a pharmaceutical composition as described herein for use in the treatment and/or prevention of at least one of pain, inflammation, bacterial infection, skin irritation.

There is also provided a use of a pharmaceutical composition as described herein for the manufacture of a medicament for the treatment and/or prevention of at least one of pain, inflammation, bacterial infection, skin irritation.

There is also provided a method of treatment and/or prevention of at least one of pain, inflammation, bacterial infection, skin irritation comprising administering to a mammal, such as a human or an animal, in need thereof an effective amount of a pharmaceutical composition as described herein.

The pharmaceutical composition described herein may be used in a pharmaceutical delivery system. Thus, there is provided a pharmaceutical delivery system comprising the pharmaceutical composition according to the appended claims, wherein said delivery system is selected from the group consisting of cream, lotion, emulsion preparation, topical liquid, aerosol solution, gel, transdermal delivery system in the form of a patch, jelly, ointment, spray, and liposome delivery system.

It will be appreciated that the pharmaceutical composition described herein may comprise a pharmaceutical excipient, diluent and/or carrier.

The pharmaceutical composition described herein may be provided as a single composition such as a composition used in a pharmaceutical delivery system described herein. The single composition may be provided with instructions for use.

Additionally or alternatively, the pharmaceutical composition described herein may be provided as a kit of parts optionally together with instructions for use. For instance, the instructions for use may be instructions for separate, sequential or simultaneous use of the components of the pharmaceutical composition.

### Further Aspects

In the current work, compositions are described comprising ropivacaine and two or more substances of the locoregional anesthetics of the amide type providing improved properties compared to a single substance of a local anesthetics of the amide type.

In particular, the invention relates to compositions comprising ropivacaine and two or more substance of the locoregional anesthetics of the amide type providing prolonged effects of local anesthesis, as well as anti-inflammatory, and anti-bacterial actions.

Unexpectedly, the pharmaceutical compositions of the present invention have been found to exhibit a rapid onset of action and/or prolonged duration compared to a pharmaceutical composition comprising a single substance of a locoregional anesthetic of the amide type.

In particular, the current invention relates to a composition comprising ropivacaine and two or more substances of the topical anesthetics of the amide type for use in medicine in general as well as to its use in treating and/or alleviating pain and/or inflammation associated with skin irritations, such as itching, rashes, and other skin sensitivities. A composition according to the present invention can be used for treating and/or alleviating a pathological condition of the skin and associated itching and pain selected from the group consisting of chicken-pox, shingles, bed sores, wounds such as superficial burn wounds, graze, insect bites, sore nipples, blisters and dendritic ulcers.

A composition according to the present invention can be used for local pre-surgical and/or post-surgical anesthesia in superficial surgical procedures such as beauty skin surgery, circumcision, and other surgical procedures of the skin.

The composition of the present invention is also suitable for use in dentistry, such as anesthesis of periodontal pockets. A preferred formulation is a gel of the present invention for use in dentistry.

The composition of the present invention is also suitable for alleviating back pain and/or joint pain and/or muscles. A preferred form for delivery of the present composition is through a transdermal patch applied to areas of the body where pain is sensed.

However, the current invention also relates to a composition comprising ropivacaine and two or more substances of the anesthetics of the amide type, for use in treating and/or alleviating pain regionally, such as during surgery, child birth, etc. In particular the composition according to the invention will be useful in cases where long surgical intervention time is expected or to achieve a better postoperative analgesia.

A composition according to the present invention comprises ropivacaine and two or more substances selected from the group of locoregional anesthetics of the amide type which are mixed and administered together.

A composition according to the present invention include locoregional anesthetics of the amide group selected from the group consisting of ropivacaine, lidocaine, prilocaine, bupivacaine, mepivacaine, tetracaine, dibucaine, articaine, etidocaine, levobupivacaine, and trimecaine.

Accordingly, the composition of the present invention preferably comprises ropivacaine, lidocaine, and prilocaine. The composition may optionally comprise hydrocortisone.

The composition of the present invention may include mixtures of ropivacaine, bupivacaine, lidocaine and prilocaine and optionally hydrocortisone.

However, any combinations of the locoregional anesthetics of the amide type are suggested but not exemplified.

The composition of the present invention comprises mixtures of ropivacaine and two or more locoregional anesthetics of the amide type at a concentration of 0.1-40% by weight. For example, the present invention includes mixtures of preferably 0.1-5% by weight of each component, more preferably 0.1-2.5% by weight of each component or 1.0-2.5% by weight of each component.

The composition of the present invention may also be selected from a group of other anesthetics.

The composition of the present invention may comprise hydrocortisone, for example, 0.1-1.0% by weight.

The composition of the present invention is administered as a formulation suitable for topical administration directly to the skin and in an amount effective to anesthetize the tissue and the specific area it is applied to. The anesthetic composition is left on the skin to exert its effects and reapplied if necessary.

The composition may alternatively be administered via injection or infusion. A lidocaine, prilocaine and ropivacaine solution may be used for epidural injection or for infusion, wherein the concentration is of 2 mg/ml, 5 mg/ml, 7.5 mg/ml or 10 mg/mL of each compound in prepared sodium chloride solution (NaCl: 3-4 mg/mL). The solution can be used to numb parts of the body during surgery or child birth, giving long lasting anaesthesia.

The composition of the present invention has a prolonged duration of anesthesis, such as up to 8 hours, and up to 6 hours or longer, such as 4-8 hours, or 4-6 hours.

The composition of the present invention has rapid onset of anesthesis, such as 30 minutes or less, such as less than 20 minutes, and 10 minutes or less, such as 10-30 minutes, 10-20 minutes or 10-25 minutes.

The composition of the present invention may be formulated into any commonly accepted topical formulation. For example, the delivery system for the composition of the present invention may be in the form of a cream, lotion, emulsion preparation, topical liquid, aerosol solution, gel, patch (transdermal delivery system), jelly, ointment, spray, liposome or liposome delivery system.

The composition of the present invention may be formulated into any commonly accepted solution for epidural injection of infusion. Commonly used excipients, solvents etc. may be used in said composition.

By a liposomal formulation is to be understood as a preparation containing active ingredients in spherical vesicles having at least one lipid bilayer.

By salve it is to be understood a highly lipid solution for the application to skin. The composition of the present invention may be applied topically and left on the skin, administered for any period of time, such as hours.

By a transdermal patch it is to be understood an adhesive patch to be placed on the skin to deliver a medication.

In a further aspect, the present invention relates to a method of preparing a composition comprising ropivacaine and one or more local anesthetics according to commonly used forms of topical formulations.

The composition of the present invention may be applied topically to the skin at any time, such as pre-surgery or post-surgery.

The composition of the present invention is also suitable for veterinary purposes.

A bed sore is by definition an ulceration of the skin and subcutaneous tissue, commonly caused by deprived blood circulation in parts of the body.

It will be appreciated that the invention is not limited by the embodiments described herein, and further modifications of the invention within the scope of the claims would be apparent to a skilled person.

The disclosure is further illustrated by the following non-limitative Examples

### Experimental Section

Compositions for topical administration comprising mixtures of ropivacaine and one or more substances selected from the group of topical anesthetics of the amide type were prepared.

The duration of anesthesis may depend on the type of topical anesthetics and amount applied, but is usually about half an hour to one hour for substances selected from the group of topical anesthesics of the amide type. For example, ropivacaine has about two times longer duration time of anesthesis compared to lidocaine. Ropivacaine also has antibacterial and anti-inflammatory properties. Lidocaine is characterised by a rapid onset of action and intermediate duration of efficacy as a topical anesthetics. Prilocaine is short- to moderate-acting local anesthetic substance and similar to but less vasodilatory than lidocaine. Bupivacaine is often administered by injections and has a long duration. Finally, hydrocortisone was used in combination with ropivacaine. Hydrocortisone is an immunosuppressive drug in the treatment of severe allergic reactions.

The pharmaceutical compositions were topically administered to intact skin of the arm and tested for anesthesis by pinpricks to a treated area of the skin. The sensation of pain was recorded for every puncture of the skin by the pin. Ropivacaine mixed with one or more substance selected from the group of local anesthetics of the amide type, such as lidocaine, prilocaine or bupivacaine provide advantageous properties. In order to increase the skin penetration of the mixtures comprising ropivacaine and to control the rate of penetration (for example a 24 hour dose) will require novel formulations. The formulations of ropivacaine formulated as a salve, by liposomal delivery, a cream, or as a water and propylene glycol solution for pre-surgical or post-surgical anesthesia and anti-inflammatory treatments will provide advantageous effects.

### EXAMPLE 1

The compositions comprising ropivacaine and one or more substance were prepared. The compositions were formulated in liposomal preparation, in a salve, or in a propylene glycol and water solution, and the active ingredients were at a 2.5% by weight or 1.0% by weight.

The compositions were applied to intact skin of the lower arm in a circle area of a diameter of 1-2 cm. The amount of the composition applied to the skin was kept constant and left on the skin during the study. The treated area of intact skin was subjected to repeated pinpricks and the sensation of pain was recorded. The sensation of pain was recorded at 20 minutes, 30 minutes, 1 hour, 2, 3, 4 and 5 hours.

The results are disclosed in table 1.

Ropivacaine was applied to skin of the lower arm and the onset of anesthesis occurred at approximately 1 hour, and the effect lasted 3 hours. In contrast, there was unexpected prolonged anesthesis of the skin treated with mixtures comprising ropivacaine mixed with one or more active substances. There is approximately a two-fold prolonged anesthesis achieved by the compositions of the present invention. Furthermore, the onset of the anesthesis is faster for compositions of the present invention. The onset is approximately 20 to 30 minutes faster for the compositions of the invention.

The strongest enhancements of duration and onset of anesthesia was observed for the composition comprising ropivacaince, lidocaine and prilocaine at 2.5% by weight of each substance in a liposomal formulation.

TABLE 1
The duration and onset of anesthesis for
compositions of the present invention.
The mark x in the table indicates anesthesis and
loss of pain sensation in skin area where the composition
was applied and subsequently pinpricked at indicated times.

| | Studied effect and | | | | | |
|---|---|---|---|---|---|---|
| | its duration (hrs) | | | | | |
| Composition | 0.3-0.5¹ | 1.0 | 2.0 | 3.0 | 4.0 | 5.0 |
| Ropivacaine | | x | x | x | | |
| I Ropivacaine + lidocaine | x | x | x | x | | |
| (2.5% + 2.5%) | | | | | | |
| formulation: liposomal | | | | | | |
| II Ropivacaine + prilocaine | x | x | x | x | | |
| (2.5% + 2.5%) | | | | | | |
| formulation: liposomal | | | | | | |
| III Ropivacaine + lidocaine + | x | x | x | x | x | x |
| prilocaine (2.5% + 2.5% + 2.5%) | | | | | | |
| formulation: liposomal | | | | | | |
| IV Ropivacaine + hydrocortisone | | x | x | x | | |
| (2.5% + 1%) | | | | | | |
| formulation: salve | | | | | | |
| V Ropivacaine + | x | x | x | | | |
| prilocaine (2.5% + 2.5%) | | | | | | |
| formulation: | | | | | | |
| propylene glycol + | | | | | | |
| H₂O (50:50) solution | | | | | | |

¹The onset of anesthesis occurred at 0.5 hour for compositions formulated in salve. The onset of anesthesis occurred at 0.3 hour for compositions formulated in liposomal and propylene glycol + H₂O solution (50:50).

### Materials and Methods

Ropivacaine, lidocaine, prilocaine, tetracaine, bupivacaine, hydrocortisone were purchased from Sigma-Aldrich AB, Stockholm, Sweden.

Propylene glycol/H₂O solution (50:50) was purchased from Apotek AB, Stockholm, Sweden.

### Epidural Administration or Infusion

As stated above under the Summary, Lazăr et al. reported that a longer lasting effect was obtained with a combination of lidocaine and ropivacaine solution being used epidurally, as compared to a solution containing ropivacaine alone, A combination of anesthetics thus leads to a longer duration of anesthesia also epidurally. Thus, the same results relating to duration and onset of anesthesia as the results shown above for topical application, are expected for the pharmaceutical composition formulated as a solution for epidural injection or for infusion according to the appended claims,

1. A pharmaceutical composition for alleviation of pain and/or skin inflammation comprising:
(i) ropivacaine, and
(ii) two or more anesthetics of amide type.

2. A pharmaceutical composition according to claim 1, wherein the anesthetic of amide type is selected from the group consisting of bupivacaine, lidocaine, mepivacaine, prilocaine, and tetracaine.

3. A pharmaceutical composition according to claim 1, wherein said pharmaceutical composition comprises from 1% by weight to 5% by weight of ropivacaine and from 1% by weight to 5% by weight of the two or more anesthetics of amide type.

4. A pharmaceutical composition according to claim 1, wherein said pharmaceutical composition comprises 2.5% by weight of ropivacaine, 2.5% by weight of prilocaine and 2.5% by weight of lidocaine.

5. A pharmaceutical composition according to claim 1, further comprising hydrocortisone.

6. (canceled)

7. A method for the treatment and/or prevention of at least one of pain, inflammation, bacterial infection, or skin irritation in a mammal, comprising administering the composition according to claim 1 to the mammal.

8. A pharmaceutical composition according to claim 1, wherein said pharmaceutical composition is a topical pharmaceutical composition.

9. A pharmaceutical composition according to claim 1, wherein said pharmaceutical composition is a solution for epidural injection or for infusion.

10. A method for the manufacture of a medicament for the treatment and/or prevention of at least one of pain, inflammation, bacterial infection, or skin irritation, comprising combining (i) ropivacaine, and (ii) two or more anesthetics of amide type.

11. A method for the treatment and/or prevention of at least one of pain, inflammation, bacterial infection, or skin irritation comprising administering to a mammal, in need thereof, an effective amount of a pharmaceutical composition as defined in claim 1.

12. A pharmaceutical delivery system comprising the pharmaceutical composition according to claim 1, wherein said delivery system is selected from the group consisting of cream, lotion, emulsion preparation, topical liquid, aerosol solution, gel, transdermal delivery system, jelly, ointment, spray and liposome delivery system.

13. The method of claim 11, wherein the mammal is a human or an animal.

14. A pharmaceutical composition for alleviation of pain and/or skin inflammation comprising:
(i) bupivacaine, and
(ii) two or more anesthetics of amide type.

15. A pharmaceutical composition for alleviation of pain and/or skin inflammation comprising:
(i) prilocaine, and
(ii) two or more anesthetics of amide type.
